(19) United States
(12) Patent Application Publication
(10) Pub. No.: US 2010/0249929 A1
(43) Pub. Date: Sep. 30, 2010

(54) ADJUSTABLE PATELLA LIGAMENT FOR ACL REPAIR

(75) Inventors: Arthur Kurz, Cary, IL (US); Raphael Diaz, Denver, CO (US)

Correspondence Address: HOLLAND & HART, LLP, P.O BOX 8749, DENVER, CO, 80201, US

(73) Assignee: JOINT RESTORATION FOUNDATION, Centennial, CO (US)

(21) Appl. No.: 12/730,572

(22) Filed: Mar. 24, 2010

Related U.S. Application Data
(60) Provisional application No. 61/162,687, filed on Mar. 24, 2009.

(51) Int. Cl. A61F 2/08

(52) U.S. Cl. 623/13.14; 623/13.11

(57) ABSTRACT

There are disclosed methods of performing an ACL repair. In one embodiment, the method includes providing a patella ligament with two native bone blocks and the patella ligament has a length greater than a native ACL. The length of the patella ligament is shortened to a desired length for an ACL repair while maintaining at least one native interface between the patella ligament and the native bone blocks. There are disclosed adjustable patella ligaments for ACL repair. In one embodiment, the ligament includes native bone blocks with the length between shortened from a length greater than a native ACL. A securing member is attached to one of the blocks and the patella ligament. At least one block is attached to the patella ligament by a native interface. Other embodiments are also disclosed.

### REFERENCE TO PENDING PRIOR PATENT APPLICATION

This application claims the benefit under 35 U.S.C. 119 (e) of U.S. Provisional Patent Application No. 61/162,687, filed Mar. 24, 2009 by Arthur Kurz, et al., for “ADJUSTABLE PATELLA LIGAMENT FOR ACL REPAIR,” which patent application is hereby incorporated herein by reference.

### BACKGROUND

At one time the gold standard for ACL replacement was the patella ligament. The primary reason surgeons moved away from the patella ligament had to do with supply. Suppliers could provide a larger quantity of non-bone tendons (Semi-Tendonosus, Gracilis, and Tibialis) to meet the demand. As fixation devices improved, surgical procedures changed and surgeons increasingly embraced the use of non-bone tendons. Due to the comfort with non-bone tendons and the associated fixation devices, what was once the gold standard allograft is now associated with a procedure that causes some surgical challenges.

### SUMMARY OF THE INVENTION

In an embodiment, there is provided a method of providing an adjustable patella ligament for ACL repair, the method including providing a patella ligament with two native bone blocks, and the patella ligament having a length greater than a native ACL; and shortening the length of the patella ligament, to a desired length for an ACL repair, while maintaining at least one native interface between the patella ligament and the native bone blocks. In one embodiment, the method further includes attaching each of the native bone blocks to bone tunnels. In an embodiment, the method may include flipping one of the bone blocks against the patella ligament. In one embodiment, the method may include detaching one of the bone blocks from the patella ligament; and the step of shortening the length of the patella ligament may include reattaching the native bone block to the patella ligament. In another embodiment, the method may include providing one of the native bone blocks having a length sized to fill the bone tunnel.

In another embodiment, there is provided an adjustable patella ligament for ACL repair, the ligament having native bone blocks with a length of the ligament therebetween shortened from a length greater than a native ACL; a securing member attached to one of the native bone blocks and the patella ligament; and at least one of the native bone blocks attached to the patella ligament by a native interface therebetween. In one embodiment, the securing member may include suture attaching the native bone block to the patella ligament. In an embodiment, the patella ligament with the native bone block is detached from the native interface to the patella ligament.

In still another embodiment, there is provided adjustable patella ligament for ACL repair, the ligament comprising native bone blocks with a length of the ligament therebetween shortened from a length greater than a native ACL; a securing member attached to one of the native bone blocks and the patella ligament; and at least one of the native bone blocks attached to the patella ligament by a native interface therebetween.

Other embodiments are also disclosed.

### BRIEF DESCRIPTION OF THE DRAWINGS

Illustrative embodiments of the invention are illustrated in the drawings, in which:

FIG. 1 illustrates a bone-tendon-bone ligament as recovered from a donor with bone plugs carved or otherwise manipulated;

FIG. 2 illustrates the bone-tendon-bone ligament of FIG. 1 with bone blocks repositioned against the ligament in order to shorten the inter-bone tendon length as well as the total length of the bone-tendon-bone ligament from opposed ends;

FIG. 3 illustrates one exemplary embodiment of the bone-tendon-bone ligament of FIGS. 1 and 2 with an interference fixation at each of the bone blocks;

FIG. 4 illustrates a bone-tendon-bone ligament with bone blocks joined at native interfaces to the ligament;

FIG. 5 illustrates the bone-tendon-bone ligament of FIG. 4 with the bone block cut away from the ligament and the cut positioned at the native interface between the ligament and the bone block;

FIG. 6 illustrates the bone-tendon-bone ligament of FIGS. 4 and 5 with the cut-off native bone block reattached to the ligament in order to provide a shortened length of the inter-bone tendon length as well as the overall length of the bone-tendon-bone ligament;

FIG. 7 illustrates a bone-tendon-bone ligament with one bone block longer in length than the other bone block; and

FIG. 8 illustrates the bone-tendon-bone ligament of FIG. 7 after one of the bone blocks has been flipped and attached with suture onto the ligament.

### DETAILED DESCRIPTION

There are several challenges in using the patella ligament for ACL replacement. These challenges include the fact that the native ACL has a length of about to 3.5 cm to about 3.8 cm. Over 50 percent of patellar ligament inter-bone length is over 4.0 cm. This means than the bone block and tendon length often exceed the length of the bone tunnel.

The fixation of tendon to bone is likely the best test of success in ACL repair. This fixation may be more important than tendon strength and stiffness. The strength and stiffness of the patella ligament is significantly better than a native ACL. Although fixation devices have improved significantly over the years, nothing has replaced the bone-tendon interface (i.e., the native interface) found on the bone-tendon-bone patella ligament.

It's well established that there is accelerated healing with a cancellous bone-bone interface. The bone-tendon interface of the patella ligament is already established. The cancellous patella ligament bone blocks interface with the bone tunnel and accelerate incorporation.

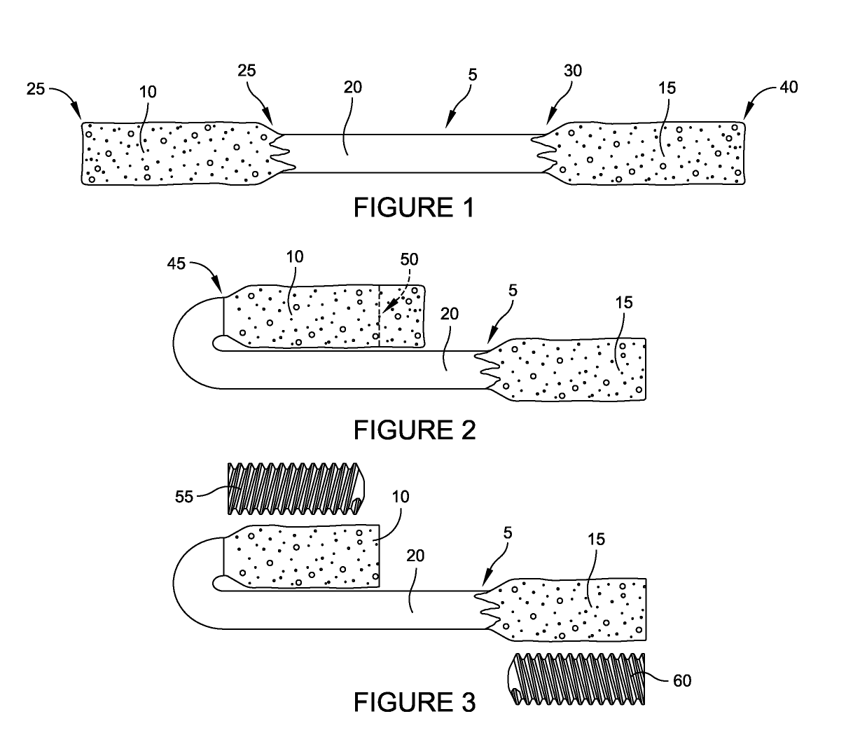

Referring to FIG. 1, there is shown a bone-tendon-bone 5 (BTB 5) as recovered from a donor with bone plugs carved or otherwise manipulated. In one embodiment, the donor of BTB 5 may be a human. In another embodiment, the donor of BTB 5 may be an animal other than a human. BTB 5 includes bone blocks 10, 15 joined together by a ligament 20 at native interfaces 25, 30. Generally, bone blocks 10, 15 are cancellous bone blocks with a patellar tendon. BTB 5 has an overall length extending between opposed ends 35, 40. In an embodiment, bone blocks 10, 15 may be pre-sized for particular bone tunnels for a patient.

Referring now to FIG. 2, there is shown BTB 5 with bone blocks 10 repositioned against ligament 20 in order to shorten the inter-bone tendon length as well as the total length of BTB 5 from opposed ends 35, 40. By repositioning, or “flipping,” BTB 5 with respect to ligament 20, the overall length of BTB 5 may be selectively adjusted without separation of native interfaces 25, 30 joining together bone blocks 10, 15 to ligament 20. These native interfaces 25, 30 may provide additional overall strength for BTB 5.

In one embodiment, the lengths of bone blocks 10, 15 and the length of ligament 20 may permit desired use in an ACL reconstruction or other procedure with only bone block 10 being flipped. As illustrated in FIG. 2, bone block 10 is flipped with an end 45 (which is opposite to native interface 25) positioned as far as possible from native interface 50 of bone block 15. This configuration may provide good strength of BTB 5 while shortening the useful length of ligament 20. Potential options to increase the useful length of ligament 20 may include resecting bone block 10 to shorten its length, such as at location 50. In another embodiment, the useful length of ligament 20 may be decreased by positioning end 45 of bone block 10 closer to native interface 25. This configuration leaves an excess portion of ligament at the flip site of end 35. However, depending on the type of fixation employed at the bone block 10 to a bone tunnel, the excess portion may not create any significant issues.

Referring now to FIG. 3, there is shown one exemplary embodiment of an interference fixation at each of bone blocks 10, 15. In this embodiment, interference screws 55, 60 may be used to fixedly secure bone blocks 10, 15 to respective bone tunnels (not shown.) In other embodiments, fixation hardware may be similar or different at bone blocks 10, 15. Some examples include, but are not limited to, fixation pins, expandable members, and suture loops.

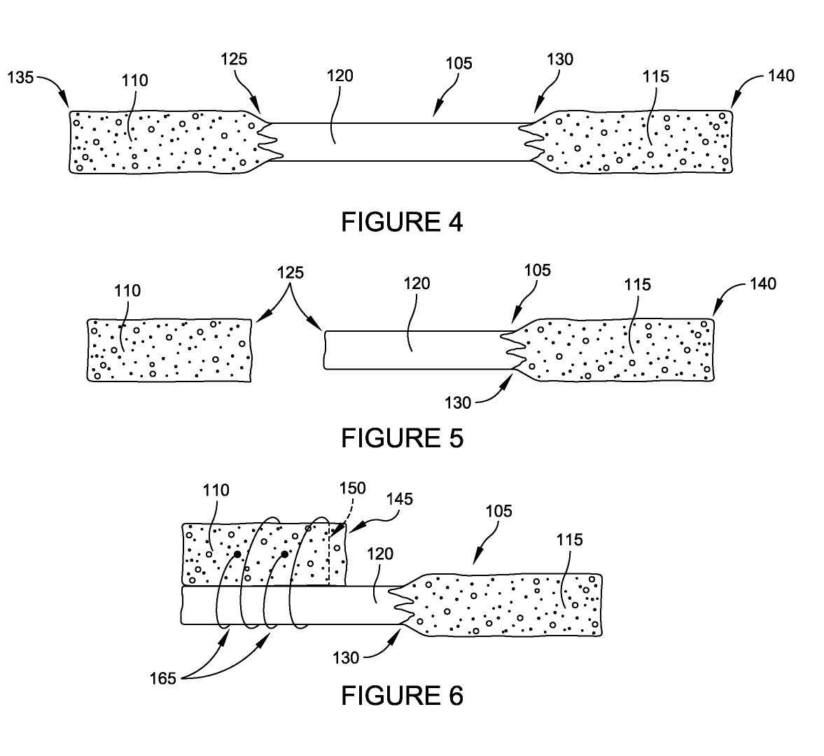

Referring now to FIGS. 4-6, and in another embodiment, there is shown a BTB 105, which may be similar to BTB 5, as recovered from a donor. Referring to FIG. 4, BTB 105 may include bone blocks 110, 115 joined together by a ligament 120 at native interfaces 125, 130. BTB has a native overall length extending between ends 135, 140.

Referring to FIG. 5, and in one embodiment, bone block 110 may be cut away from ligament 120, and this cut may be positioned at native interface 125. In another embodiment, bone block 110 may be cut away at another position of ligament 120 in order to further shorten the overall length of BTB 105.

Looking at FIG. 6, and in an embodiment, bone block 110 may be attached to ligament 120 in order to provide a shortened length of the inter-bone tendon length as well as a shortened overall length of BTB 105. In one embodiment, stitching 165 may provide this attachment of bone block 110 to ligament 120. Similar to the “flipped” construct described herein above, end 145 of bone block 110 may be positioned closer to, or away from, native interface 130 of bone block 115 in order to provide a desired length of ligament 120 between end 145 of bone block 110 and native interface 130 of bone block 115. In one embodiment, suture material 165 may be used to fixedly attach bone block 110 to ligament 120.

Bone block 110 may be resized by resection of portion 150 or at other portions inasmuch as bone block 110 is removed prior to reattachment to ligament 120. Native interface 130 may provide BTB 105 with enhanced strength, and overall simplicity as opposed to attaching two bone block portions to ligament 120.

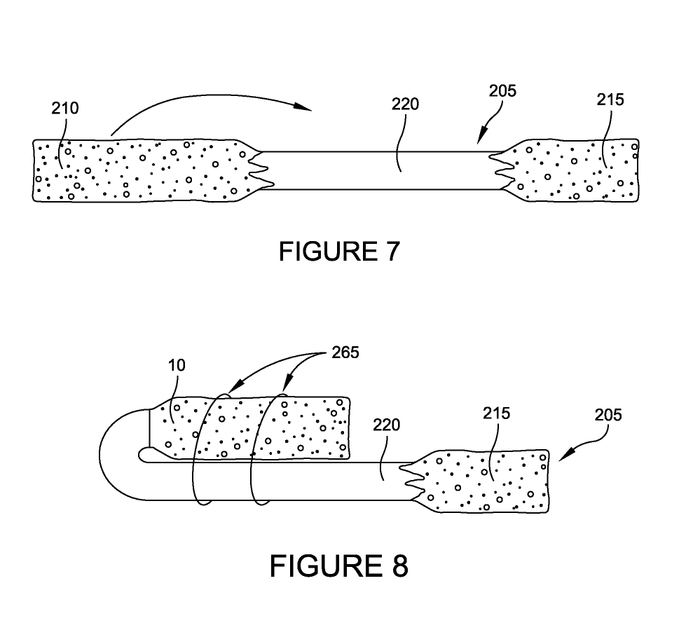

Referring now to FIGS. 7 and 8, and in one embodiment, there is shown a BTB 205 having a bone block 210 with a longer length than bone block 215. For example, bone block 210 may be a tibial bone block with dimensions of approximately 40 mm×10 mm and bone block 215 may be a patella bone block with dimensions of approximately 25 mm×10 mm. FIG. 8 illustrates BTB 205 after bone block 210 has been flipped and attached with suture 265 onto ligament 220. The size of bone block 210 is chosen to provide a void filler allograft with the flipped portion of bone block 210. For an ACL procedure, bone block 210 and tendon 220 along side bone block 210 provide support to prevent creeping in the bone tunnel. For fixation, the tibial side may be fixated with, but is not limited to, 2×20 mm ACL screws and the femoral side may be fixated using standard fixation or other fixation. Using BTB 205, a surgeon may be provided with combined benefits of filling the bone tunnel with bone and having a tendon with an appropriate inter bone block ligament length.

The BTB structures disclosed herein may be used to provide joint-line fixation. In one embodiment, joint-line fixation includes positioning the end of a bone block at the end of the bone tunnel at the interface into the opening of the joint. For example, the patella ligament (BTB 5, 105, or 205) may be positioned with the bone block at the bone tunnel interface with the opening of the joining to allow for joint-line fixation. Joint-line fixation reduces tunnel widening and minimizes micromotion of the tendon within the tunnel.

The BTB structures disclosed here may also be used together with other materials for filling and securing the bone tunnel. This filling and securing the bone tunnel reduces tunnel widening and minimizes micromotion of the tendon within the tunnel. For example, if bone blocks cannot be placed at the joint line, the proximal end of the tunnel may be filled with a pre-drilled cancellous core or cancellous bone sponge.

Filling and securing the bone tunnel also may provide accelerated tendon-bone healing within the tunnel. Furthermore, filling and securing the bone tunnel may reduce tendon degradation from friction within the tunnel. Filling and securing the bone tunnel may also provide accelerated healing with the provided bone-bone interface.

What is claimed is:

1. A method of performing an ACL repair, the method comprising:
providing a patella ligament with two native bone blocks, and the patella ligament having a length greater than a native ACL; and
shortening the length of the patella ligament, to a desired length for an ACL repair, while maintaining at least one native interface between the patella ligament and the native bone blocks.

2. A method in accordance with claim 1, further including attaching each of the native bone blocks to bone tunnels.

3. A method in accordance with claim 1, further including flipping one of the bone blocks against the patella ligament.

4. A method in accordance with claim 3, further including attaching at least one of the native bone blacks to a bone tunnel with at least one native interface disposed away from a joint between bone tunnels.

5. A method in accordance with claim 4, further including positioning an end of one of at least one of the bone blocks in alignment with a bone surface at the joint so as to provide a joint-line fixation.

6. A method in accordance with claim 1, further including detaching one of the bone blocks from the patella ligament.

7. A method in accordance with claim 6, wherein the step of shortening the length of the patella ligament includes reattaching the native bone block to the patella ligament.

8. A method in accordance with claim 1, further including providing one of the native bone blocks having a length sized to fill the bone tunnel.

9. A method in accordance with claim 1, further including positioning an end of one of at least one of the bone blocks in alignment with a bone surface at a joint so as to provide a joint-line fixation.

10. A method in accordance with claim 1, further including filling an end of at least one of the bone tunnels adjacent a joint with a fill material.

11. A method in accordance with claim 10, wherein the fill material is pre-drilled cancellous bone.

12. A method in accordance with claim 10, wherein the fill material is a bone sponge.

13. A method of performing an ACL repair, the method comprising:
providing a patella ligament having a length between two native bone blocks greater than a length across a joint between two bone surfaces having bone tunnels therein;
flipping at least one of the bone blocks against the patella ligament; and
attaching the two bone blocks in the bone tunnels while maintaining at least one native interface between the patella ligament and the bone blocks so as to dispose the at least one native interface of the at least one of the bone blocks flipped against the patella ligament away from the joint.

14. A method in accordance with claim 13, further including providing one of the native bone blocks having a length sized to fill the bone tunnel.

15. A method in accordance with claim 13, further including detaching one of the bone blocks from the patella ligament.

16. A method in accordance with claim 13, wherein the step of shortening the length of the patella ligament includes reattaching the native bone block to the patella ligament.

17. A method in accordance with claim 13, further including providing one of the native bone blocks having a length sized to fill the bone tunnel.

18. A method in accordance with claim 13, further including positioning an end of one of at least one of the bone blocks in alignment with a bone surface at the joint so as to provide a joint-line fixation.

19. A method in accordance with claim 14, further including filling an end of at least one of the bone tunnels adjacent the joint with a fill material.

20. A method in accordance with claim 19, wherein the fill material is pre-drilled cancellous bone.

21. A method in accordance with claim 19, wherein the fill material is a bone sponge.

22. An adjustable patella ligament for ACL repair, the ligament comprising:
native bone blocks with a length of the ligament therebetween shortened from a length greater than a native ACL;
a securing member attached to one of the native bone blocks and the patella ligament; and
at least one of the native bone blocks attached to the patella ligament by a native interface therebetween.

23. An adjustable patella ligament according to claim 22, wherein the securing member includes suture attaching the native bone block to the patella ligament.

24. An adjustable patella ligament according to claim 23, wherein the native bone block attached to the patella ligament with both suture and the native interface.

25. An adjustable patella ligament according to claim 24, wherein one of the bone blocks is flipped against the patella ligament.

26. An adjustable patella ligament according to claim 25, wherein the one of the bone blocks flipped against the patella ligament is attached with at least one native interface disposed away from the other one of the bone blocks.

27. An adjustable patella ligament according to claim 24, wherein both of the bone blocks are flipped against the patella ligament.

28. An adjustable patella ligament according to claim 27, wherein both of the bone blocks flipped against the patella ligament are attached with the one native interface disposed away from the other one of the bone blocks.

29. An adjustable patella ligament according to claim 23, wherein the native bone block is attached to the patella ligament with suture and the native bone block is detached from the native interface of the patella ligament.

30. An adjustable patella ligament according to claim 22, wherein the patella ligament includes one of the native bone blocks detached from the native interface.
